(19) United States
(12) Patent Application Publication
(10) Pub. No.: US 2025/0326710 A1
(43) Pub. Date: Oct. 23, 2025

(54) METHOD FOR CHLORINATING BENZALDEHYDE OXIMES

(71) Applicant: BAYER AKTIENGESELLSCHAFT, Leverkusen (DE)

(72) Inventors: Andreas Rembiak, Leverkusen (DE); Anton Lishchynskyi, Leverkusen (DE)

(21) Appl. No.: 18/865,187

(22) PCT Filed: May 11, 2023
(86) PCT No.: PCT/EP2023/062623

§ 371 Date: Nov. 12, 2024

(30) Foreign Application Priority Data
May 13, 2022 (EP) 22173397.5

(51) Int. Cl. C07C 259/02

(52) U.S. Cl. CPC C07C 259/02

(57) ABSTRACT

The present invention relates to a novel process for preparing chlorobenzaldehyde oximes of the general formula (I).

The present invention relates to a novel process for preparing chlorobenzaldehyde oximes of the general formula (I).

Chlorobenzaldehyde oximes of the general formula (I) are important precursors of active agrochemical ingredients (cf. WO 2018/228985) and active pharmaceutical ingredients (e.g. DNA-binding agents: Woods, Craig R. et al. Bioorganic & Medicinal Chemistry Letters, 12 (18), 2647-2650, 2002).

Numerous chlorination methods are described in the prior art; for example, WO 2004/29066 and Industrial Crops & Products 2019, 140, 111706 teach the preparation of chlorobenzaldehyde oximes by reacting oximes with N-chlorosuccinimide (NCS) and subsequent aqueous work-up (extraction with EtOAc/H₂O). However, only small amounts (2.45 g) of the chlorobenzaldehyde oximes obtained were isolated in solid form in the process described. In principle, the isolation of chlorobenzaldehyde oximes in solid form on an industrial scale is undesirable, however, since chlorobenzaldehyde oximes are often high-energy compounds which exhibit a high tendency to decompose. The process described in WO 2004/29066 uses dimethylformamide (DMF) as solvent. It is known, however, that the use thereof as solvent on an industrial scale may be problematic. This is due to the strongly exothermic reaction between DMF and the chlorinating agent, which then possibly proceeds in an uncontrolled manner. (OPRD 2020, 24, 1586; Bull. Chem. Soc. Jpn. 1994, 67, 156).

The use of chlorine gas for chorinating oximes is described, for example, in the Journal of Organic Chemistry 1971, 36, 2146. In this case, the reactions are carried out exclusively in chloroform or dichloromethane at high dilutions, which is not suitable for large-scale industrial synthesis and affords the chlorobenzaldehyde oximes in moderate to low yields of 3-80%. Preferred chlorination under basic conditions by adding triethylamine as described results in partial decomposition of the chloroxime compound. In addition, the Journal of Heterocyclic Chemistry; 2012, 49, 621 teaches the use of chlorine gas in methanol as solvent under mildly basic reaction conditions by adding sodium carbonate. In addition to the formation of safety-relevant amounts of gaseous carbon dioxide, the product may again decompose under basic conditions. The hydrogen chloride released during chlorination can also lead to formation of toxic compounds by reaction with the solvent. The use of methanol as solvent also prevents direct use of the product solutions without changing solvents in many chemical reactions. In addition to safety-relevant limitations due to the high energy of such chlorine compounds and the preferred uses of dilute systems, the change of solvents also reduces the efficiency and the sustainability of the process.

The Journal of Enzyme Inhibition and Medicinal Chemistry; vol. 31; issue 6; (2016); pp. 964-973 teaches the chlorination of oximes using trichloroisocyanuric acid (TCCA) with triethylamine as base. Although DMF is not used as solvent here, it has been observed that the chloroximes tend to degrade in the basic medium due to the formation of nitrile oxides, which may lead to yield losses (for example dimerization of the nitrile oxides to furoxanes): “Kinetics and Mechanism of 1,3-Dipolar Cycloadditions” by Prof. Dr. R. Huisgen, Angew. Chem. 1963, 75, 742-754, page 751; “Fragmentation of Nitrile Oxides with Triethylamine” Tetrahedron Lett. 1983, 24, 4377-4380). In addition, the use of trichloroisocyanuric acid (TCCA) produces larger amounts of solid isocyanuric acid as waste product, which is detrimental to the process in terms of sustainability and waste management.

The invention was therefore based on the object of providing a process for chlorinating benzaldehyde oximes which, on the one hand, dispenses with DMF or chlorinated alkane compounds as solvent and the use of low atom-economy and waste-heavy chlorinating agents such as trichloroisocyanuric acid or N-chlorosuccinimide and, on the other hand, does not entail the losses in yield caused by relatively strong bases such as triethylamine, but at the same time is cost-effective and can be used on an industrial scale. The chlorobenzaldehyde oximes should be obtained in high yields in this case and may be used as a solution without the mandatory necessity of a solvent exchange in varied chemical reactions by suitable selection of the solvent.

The object was achieved according to the invention by a process for preparing chlorobenzaldehyde oximes of the general formula (I)

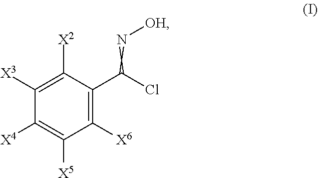

in which
X² is H, C₁-C₄ alkyl, C₁-C₄ fluoroalkyl, C₁-C₄ fluoroalkoxy, C₁-C₄ alkoxy, fluorine, CN,
X³ is H, C₁-C₄ alkyl, C₁-C₄ fluoroalkyl, C₁-C₄ fluoroalkoxy, C₁-C₄ alkoxy, fluorine, chlorine, CN,
X⁴ is H, C₁-C₄ alkyl, C₁-C₄ fluoroalkyl, C₁-C₄ fluoroalkoxy, C₁-C₄ alkoxy, fluorine, CN,
X⁵ is H, C₁-C₄ alkyl, C₁-C₄ fluoroalkyl, C₁-C₄ fluoroalkoxy, CI-C₄ alkoxy, fluorine, chlorine, CN,
X⁶ is H, C₁-C₄ alkyl, C₁-C₄ fluoroalkyl, C₁-C₄ fluoroalkoxy, C₁-C₄ alkoxy, fluorine, CN,
   characterized in that the compounds of the general formula (II)

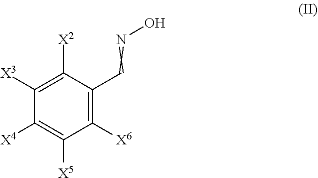

in which
X² to
X⁶ have the meanings stated above,
   are converted to compounds of the general formula (1) with the aid of chlorine gas (Cl₂).

In a preferred embodiment, an amide base is added to the reaction mixture in addition to chlorine gas, in order to convert the compounds of the formula (II) to compounds of the general formula (I).

In a particularly preferred embodiment, catalytic amounts of an amide base are added to the reaction mixture in addition to chlorine gas, in order to convert the compounds of the formula (II) to compounds of the general formula (I).

Preferred definitions of the radicals for the compounds of the general formulae (I) and (II) are as follows:
X² is H, methyl, trifluoromethyl, difluoromethyl, difluoromethoxy, trifluoromethoxy, fluorine, methoxy, CN,
X³ is H, methyl, trifluoromethyl, difluoromethyl, difluoromethoxy, trifluoromethoxy, fluorine, chlorine, methoxy, CN,
X⁴ is H, methyl, trifluoromethyl, difluoromethyl, difluoromethoxy, trifluoromethoxy, fluorine, methoxy, CN,
X⁵ is H, methyl, trifluoromethyl, difluoromethyl, difluoromethoxy, trifluoromethoxy, fluorine, chlorine, methoxy, CN,
X⁶ is H, methyl, trifluoromethyl, difluoromethyl, difluoromethoxy, trifluoromethoxy, fluorine, methoxy, CN.

Particularly preferred definitions of the radicals for the compounds of the general formulae (I) and (II) are as follows:
X² is H,
X³ is H, methyl, trifluoromethyl, difluoromethyl, fluorine, chlorine, methoxy, CN,
X⁴ is fluorine, H,
X⁵ is H, methyl, trifluoromethyl, difluoromethyl, fluorine, chlorine, methoxy, CN,
X⁶ is H.

Very particularly preferred definitions of the radicals for the compounds of the general formulae (I), (II) are as follows:
X² is H,
X³ is H, fluorine,
X⁴ is H, fluorine,
X⁵ is H, fluorine,
X⁶ is H.

Most preferred definitions of the radicals for the compounds of the general formulae (I) and (II) are as follows:
X² is H,
X³ is fluorine,
X⁴ is H,
X⁵ is fluorine,
X⁶ is H.

The compounds of the formula (I) may be present as mixtures of geometric isomers:

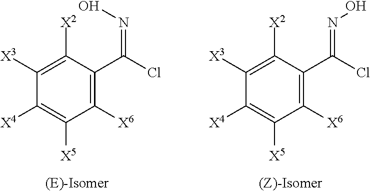

The ratio between E and Z isomers varies.

### Elucidation of the Processes and Intermediates

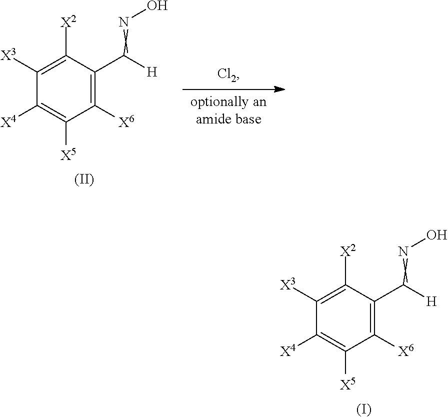

Process for preparing chlorobenzaldehyde oximes of the formula (I), characterized in that the compounds of the general formula (II) are converted to compounds of the general formula (I) with the aid of chlorine gas (Cl₂).

Preferably, an amide base is added to the reaction mixture in addition to chlorine gas, in order to convert the compounds of the formula (II) to compounds of the general formula (I).

Particularly preferably, catalytic amounts of an amide base are added to the reaction mixture in addition to chlorine gas, in order to convert the compounds of the formula (II) to compounds of the general formula (I).

The process according to the invention has the advantage that the use of stoichiometric amounts of DMF or chlorinated alkane compounds as solvent is avoided. This minimizes the risk that the reaction proceeds in a highly exothermic and uncontrolled manner and eliminates the need for low-sustainable solvents. In addition, the use of chlorine gas eliminates the need to use N-chlorosuccinimide (NCS) or trichloroisocyanuric acid (TCCA), thus reducing waste and significantly increasing the sustainability of the process. The reaction is therefore suitable for performance on a large scale.

Amide bases suitable as catalysts are, for example, dimethylformamide (DMF), dibutylformamide (DBF), diethylformamide (DEF), or dimethylacetamide (DMAc), with dimethylformamide or dibutylformamide being preferred.

In the process according to the invention, preferably no amide base or 0.05-0.3 equivalents of an amide base are used, based on the benzaldehyde oxime (II), particularly preferably 0.1-0.3 equivalents. Preferably, 0.95-2.0 equivalents of Cl₂ are used, based on the benzaldehyde oxime (II), particularly preferably 1.0-1.5 eq.

The chlorination is usually carried out in a temperature range from −10° C. to 40° C., preferably-5° C. to 10° C., particularly preferably 0 to 10° C.

The chlorination is furthermore carried out in the presence of a solvent or diluent, preferred solvents being tetrahydrofuran, Me-THF, acetonitrile, N,N-dimethylacetamide, toluene, xylene, chlorobenzene, ethyl acetate, isopropyl acetate, methyl tert-butyl ether, cyclopentyl methyl ether, tert-amyl methyl ether or mixtures of the solvents specified.

Chlorine is introduced to the benzaldehyde oxime of the formula (II) as a gas.

Preference is given to working under anhydrous conditions. The yield is thereby increased.

### EXAMPLES

The present invention is elucidated in more detail by the examples which follow, without restricting the invention thereto.

### Measurement Methods

The products were characterized by ¹H-and/or ¹⁹F-NMR spectroscopy and/or quantitative HPLC.

The NMR spectra were determined using a Bruker Avance 400 fitted with a flow probe head (volume 60 μl). In individual cases, the NMR spectra were measured with a Bruker Avance II 600.

The quantitative HPLC determinations were measured on an HPLC Agilent HP1260 Series. The technique is based on HPLC with UV detection, an Agilent XDB C₁₈ column and evaluation with external standard (use of an external standard with reference factor). The samples, reference standard, and internal standard are dissolved in acetonitrile.

### Example 1

### Without Addition of a Catalyst in Isopropyl Acetate

25.0 g of N-(3,5-difluorobenzylidene) hydroxylamine (1.0 eq.) in 225 g of isopropyl acetate were initially charged in a 0.5L reactor equipped with KPG stirrer and gas inlet tube under a protective nitrogen gas atmosphere at 23° C. After the solution had been cooled to 15° C., 14.0 g of Cl₂ (1.36 eq.) were introduced over 1 hour with stirring (300 rpm). The temperature during the addition was kept below 14-16° C. After the metered addition of Cl₂ was complete, stirring of the reaction mixture was continued for a further 30 minutes at 15° C. The HPLC analysis showed a proportion of 100% of 3,5-difluoro-N-hydroxybenzenecarboximidoyl chloride. Subsequently, the reaction mixture was cooled to 10° C. with stirring and degassed at 300 mbar for 1 h. 257.3 g of a yellow solution of 3,5-difluoro-N-hydroxybenzenecarboximidoyl chloride in isopropyl acetate were then obtained (10.1 w/w %, 92.9% yield according to QHPLC). To characterize the product by 1H-NMR, an analytical sample was completely freed of solvent in vacuo.

¹H-NMR (401 MHZ, CDCl₃): δ (ppm)=6.84-6.89 (m, 1H), 7.37-7.45 (m, 2H), 10.86 (bs, 1H).

¹⁹F-NMR (377 MHz, CDCl₃): δ (ppm)=−109.3 (m, 2F).

### Example 2

### Without Addition of a Catalyst in a Mixture of Toluene and THF

89.2 g of a solution of N-(3,5-difluorobenzylidene) hydroxylamine (1.0 eq.) in a mixture of toluene and THF (25.5 w/w %, QHPLC) were initially charged in a 0.5L reactor equipped with KPG stirrer and gas inlet tube under a protective nitrogen gas atmosphere at 23° C. and further diluted with 138 g of toluene. After the solution had been cooled to 10° C., 18.0 g of Cl₂ were introduced over 3 hours with stirring (300 rpm). The temperature during the addition was kept below 9-12° C. After the metered addition of Ch was complete, stirring of the reaction mixture was continued for a further 30 minutes at 10° C. The HPLC analysis showed a proportion of 100% of 3,5-difluoro-N-hydroxybenzenecarboximidoyl chloride. Subsequently, the reaction mixture was cooled to 0° C. with stirring and degassed at 100 mbar for 2 h. 229.7 g of a yellow solution of 3,5-difluoro-N-hydroxybenzenecarboximidoyl chloride in a mixture of toluene and THF were then obtained (9.7 w/w %, 80.5% yield according to QHPLC).

### Example 3

### With Catalyst in a Mixture of Toluene and THF

695 g of a solution of N-(3,5-difluorobenzylidene) hydroxylamine (1.0 eq.) in a mixture of toluene and THF (21.2 w/w %, QHPLC) were initially charged in a 1L reactor equipped with KPG stirrer and gas inlet tube under a protective nitrogen gas atmosphere at 23° C. and further diluted with 41.7 g of THF and also 13.7 g (0.2 eq.) of dimethylformamide (DMF) were added. After the solution had been cooled to 0° C., 77.7 g (1.17 eq.) of Cl₂ were introduced over 1.5 hours with stirring (300 rpm). The temperature during the addition was kept below 10° C. After the metered addition of Cl₂ was complete, stirring of the reaction mixture was continued for a further 30 minutes at 10° C. The HPLC analysis showed a proportion of 100% of 3,5-difluoro-N-hydroxybenzenecarboximidoyl chloride. Subsequently, the reaction mixture was cooled to 0° C. with stirring and degassed at 50 mbar for 1 h. The product solution was subsequently washed with 130 g of a 5% aqueous sodium chloride solution at 0° C. and the phases separated at 20° C. The organic product-containing phase, still containing water, was then azeotropically dried at 45° C. and 85 mbar. After addition of 258 g of toluene, 740.0 g of a pale yellow clear solution of 3,5-difluoro-N-hydroxybenzenecarboximidoyl chloride in a mixture of toluene and THF were obtained (22.3 w/w %, 91.9% yield according to QHPLC).

### Example 4

### With Catalyst in a Mixture of Chlorobenzene and THF

46.1 g of N-(3,5-difluorobenzylidene) hydroxylamine (1.0 eq.) in 145 g of chlorobenzene and 36.0 g of tetrahydrofuran were initially charged in a 0.5L reactor equipped with KPG stirrer and gas inlet tube under a protective nitrogen gas atmosphere at 23° C. and then 5.0 g of dibutylformamide (DBF, 0.1 eq.) were added. After the solution had been cooled to 0° C., 23.0 g of Cl₂ (1.2 eq.) were introduced over 40 minutes with stirring (300 rpm). The temperature during the addition was kept below 17° C. After the metered addition of Cl₂ was complete, stirring of the reaction mixture was continued for a further 30 minutes at 10° C. The HPLC analysis showed a proportion of 100% of 3,5-difluoro-N-hydroxybenzenecarboximidoyl chloride. Subsequently, the reaction mixture was cooled to 0° C. with stirring and degassed at 50 mbar for 1 h. 242.2 g of a yellowish clear solution of 3,5-difluoro-N-hydroxybenzenecarboximidoyl chloride in a mixture of chlorobenzene and THF were then obtained (21.3 w/w %, 91.7% yield according to QHPLC).

1. A method for preparing chlorobenzaldehyde oximes of general formula (I) 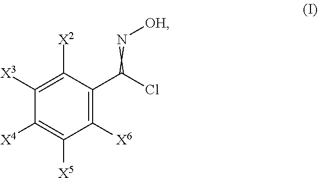
in which
X² is one of H, C₁-C₄ alkyl, C₁-C₄ fluoroalkyl, C₁-C₄ fluoroalkoxy, C₁-C₄ alkoxy, fluorine, and CN,
X³ is one of H, C₁-C₄ alkyl, C₁-C₄ fluoroalkyl, C₁-C₄ fluoroalkoxy, C₁-C₄ alkoxy, fluorine, chlorine, and CN,
X⁴ is one of H, C₁-C₄ alkyl, C₁-C₄ fluoroalkyl, C₁-C₄ fluoroalkoxy, C₁-C₄ alkoxy, fluorine, and CN,
X⁵ is one of H, C₁-C₄ alkyl, C₁-C₄ fluoroalkyl, C₁-C₄ fluoroalkoxy, C₁-C₄ alkoxy, fluorine, chlorine, and CN,
X⁶ is one of H, C₁-C₄ alkyl, C₁-C₄ fluoroalkyl, C₁-C₄ fluoroalkoxy, C₁-C₄ alkoxy, fluorine, and CN,
the method comprising:
preparing a reaction mixture comprising chloring gas (Cl₂) and compounds of [[the]] general formula (II)
in which 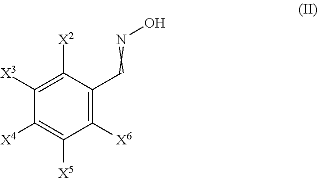
X² to
X⁶ have the meanings stated above; and
converting, in a chemical reaction, the compounds of general formula (II) to compounds of the general formula (I) with [[the]] aid of the chlorine gas (Cl₂).

2. The method according to claim 1, wherein definitions of radicals of the general formula (I) and the general formula (II) are as follows:
X² is one of H, methyl, trifluoromethyl, difluoromethyl, difluoromethoxy, trifluoromethoxy, fluorine, methoxy, and CN,
X³ is one of H, methyl, trifluoromethyl, difluoromethyl, difluoromethoxy, trifluoromethoxy, fluorine, chlorine, methoxy, and CN,
X⁴ is one of H, methyl, trifluoromethyl, difluoromethyl, difluoromethoxy, trifluoromethoxy, fluorine, methoxy, and CN,
X⁵ is one of H, methyl, trifluoromethyl, difluoromethyl, difluoromethoxy, trifluoromethoxy, fluorine, chlorine, methoxy, and CN,
X⁶ is one of H, methyl, trifluoromethyl, difluoromethyl, difluoromethoxy, trifluoromethoxy, fluorine, methoxy, and CN.

3. The method according to claim 1, wherein the definitions of the radicals of the general formula (I) and the general formula (II) are as follows:
X² is H,
X³ is one of H, methyl, trifluoromethyl, difluoromethyl, fluorine, chlorine, methoxy, and CN,
X⁴ is fluorine or H,
X is one of H, methyl, trifluoromethyl, difluoromethyl, fluorine, chlorine, methoxy, and CN,
X⁶ is H.

4. The method according to claim 3, wherein the definitions of the radicals of the general formulae (I) and (II) are as follows:
X² is H,
X³ is H or fluorine,
X⁴ is H or fluorine,
X⁵ is H or fluorine,
X⁶ is H.

5. The method according to claim 4, wherein the definitions of the radicals of the general formulae (I) and (II) are as follows:
X² is H,
X³ is fluorine,
X⁴ is H,
X⁵ is fluorine,
X⁶ is H.

6. The method according to claim 1, further comprising adding an amide base to the reaction mixture during the reaction.

7. The method according to claim 1, further comprising adding catalytic amounts of an amide base to the reaction mixture during the reaction.

8. The method according to claim 6, characterized in that the amide base is dimethylformamide (DMF), dibutylformamide (DBF), diethylformamide (DEF) or dimethylacetamide (DMAc).

9. The method according to claim 6, characterized in that the amide base is dimethylformamide (DMF) or dibutylformamide (DBF).

10. The method according to claim 1, characterized in that the reaction is conducted at −10° C. to 40° C.

11. The method according to claim 10, characterized in that the reaction is conducted at −5° C. to 10° C.

12. Precess The method according to claim 6, characterized in that 0.1-0.3 equivalents of the amide base is added, based on the benzaldehyde oxime (II).

13. The method according to claim 1, characterized in that the reaction mixture comprises 1.0-1.5 equivalents of Cl₂, based on the benzaldehyde oxime (II).
